(19) United States
(12) Patent Application Publication
(10) Pub. No.: US 2019/0117179 A1
(43) Pub. Date: Apr. 25, 2019

(54) Systems And Methods For Deciding Management Strategy in Acute Ischemic Strokes Using Rotational Angiography

(71) Applicant: MG Stroke Analytics Inc., Calgary (CA)

(72) Inventor: Mayank GOYAL, CALGARY (CA)

(73) Assignee: MG Stroke Analytics Inc., Calgary (CA)

(21) Appl. No.: 16/164,887

(22) Filed: Oct. 19, 2018

Related U.S. Application Data
(60) Provisional application No. 62/575,829, filed on Oct. 23, 2017.

(51) Int. Cl. A61B 6/00

(52) U.S. Cl. CPC A61B 6/501; A61B 6/481; A61B 6/504; A61B 6/486; A61B 2090/3966; A61B 6/507; A61B 6/5217; A61B 6/469; A61B 6/463

(57) ABSTRACT

The invention relates to systems and methods for diagnosing strokes. In particular, systems and methods for acquiring timely patient status information are described that enable a physician to make diagnostic and treatment decisions relating to strokes. The systems and methods enable the efficient and quantitative assessment of arterial collaterals within the brain for aiding these decisions in the case of ischemic strokes.

### FIELD OF THE INVENTION

The invention relates to systems and methods for diagnosing strokes. In particular, systems and methods for acquiring timely patient status information are described that enable a physician to make diagnostic and treatment decisions relating to ischemic and hemorrhagic strokes. The systems and methods enable the efficient and quantitative assessment of arterial collaterals within the brain for aiding these decisions in the case of ischemic strokes.

### BACKGROUND OF THE INVENTION

Ischemic stroke is an acute disease where tissue death (infarction) within the brain of different patients will progress at different rates from the time of the ischemic event. The rate of infarction within a patient depends on a large number of physiological factors.

For the physician diagnosing and treating ischemic strokes, when a stroke patient arrives at a hospital, it is very important for the physician to obtain as much knowledge about the nature of the stroke as soon as possible in order to make an effective diagnosis and effective decisions regarding treatment. As is readily understood, time to effect diagnosis and treatment is very important as faster diagnoses will impact treatment decisions and can minimize the amount of brain tissue that is ultimately affected as a result of the stroke.

For example, in the case of an ischemic stroke, it is important for the physician to know where the vessel occlusion is, how big the occlusion is, where any dead brain tissue (termed “core”) is and, how big and where is the brain tissue that may have been affected by the ischemic event but that may potentially be saved (this tissue is termed “penumbra”).

More specifically, the penumbra is tissue around the ischemic event that can potentially stay alive for a number of hours after the event due to perfusion of this tissue by collateral arteries. That is, the collateral arteries may provide sufficient oxygen to the penumbra tissue to prevent this tissue from dying for a period of time.

When the physician has good information about the collaterals and how the collaterals may be located in and around the penumbra, treatment decisions can be made that can significantly affect patient outcomes.

Importantly, in an emergency or acute situation, the process of making a decision will consider the amount of information at a given moment in time. That is, a definitive ‘yes’ decision can be made to take action or a ‘no’ decision can be made to take no action based on the current information. In addition, a third decision choice can be made to wait for additional information. In the situation of acute stroke (and other emergency scenarios), time to make a definitive diagnostic/treatment decision must be balanced against the likelihood of a negative outcome that results simply from the delay in making a decision. In other words, the decision to wait for more information must consider what the effects of a delay in making a decision might be.

In the specific case of acute ischemic stroke, the pace or rate of neural circuitry loss in a typical large vessel supratentorial acute ischemic stroke is shown in Table 1.

TABLE 1
Estimated Pace of Neural Circuitry Loss in Typical
Large Vessel, Supratentorial Acute Ischemic Stroke (3)
Estimated Pace of Neural Circuitry Loss in Typical
Large Vessel, Supratentorial Acute Ischemic Stroke

| | Neurons | | Synapses | | Myelinated | Accelerated | |
|---|---|---|---|---|---|---|---|
| | Lost | | Lost | | Fibers Lost | Aging | |
| Per | 1.2 | billion | 8.3 | trillion | 7140 km/ | 36 | yrs |
| Stroke | | | | | 4470 miles | | |
| Per | 120 | million | 830 | billion | 714 km/ | 3.6 | yrs |
| Hour | | | | | 447 miles | | |
| Per | 1.9 | million | 14 | billion | 12 km/ | 3.1 | weeks |
| Minute | | | | | 7.5 miles | | |

| | | | | | | |
|---|---|---|---|---|---|---|
| Per | 32,000 | 230 | million | 200 meters/ | 8.7 | hours |
| Second | | | | 218 yards | | |

As can be seen, delays in making a decision in the order of only a few minutes can have a significant impact on patient outcome in terms of neural circuitry loss. Moreover, and as shown in FIGS. 1 and 2, a better outcome is significantly more likely to occur when the decision to treat is made earlier. As shown in FIG. 1, whether or not a treatment is ultimately beneficial or not may depend on when the decision to treat is made. As shown in FIG. 1, treatment decision times A, B, C, D will each have a different affect on the relative number of neurons that could be saved. That is, if a treatment decision is made at time A (i.e. an earlier time), if it is assumed that the pace of neural circuitry loss is linear (assumed only for this example), a greater number of neurons can be saved. As the time of making the treatment decision is delayed, the likelihood of the treatment being beneficial will decrease until it is uncertain whether the treatment will be beneficial (i.e. at times B and C) or where there is a high likelihood that the treatment will be of no value (i.e. at time D).

Further, FIG. 2 illustrates the effect of time to reperfusion and good clinical outcome for observed cases where the abscissa shows time from stroke to reperfusion and the ordinate shows the probability of the patient achieving a post-treatment mRS score of 0-2. Table 2 shows the time to reperfusion and good clinical outcome for the data of FIG. 2.

TABLE 2
Time to Reperfusion and Good Clinical Outcome

| | Risk Ratio | 95% CI | p-value |
|---|---|---|---|
| Time to Reperfusion | 0.86 | 0.78-0.95 | P = 0.0045 |
| (every 30 minutes) | | | |

At the present time, in many treatment centers, when a stroke patient arrives, the assessment protocol is generally as follows:
a. Conduct a CT scan of the head to rule out or look for evidence of a hemorrhagic stroke.
b. Conduct a CT angiogram (CTA) to locate the site of vessel occlusion.
c. Conduct a CT perfusion (CTP) study to create perfusion maps that provide the physician with information about various parameters including cerebral blood flow, cerebral blood volume and mean transit time.

As is known, each of these generalized steps will be affected by a large number of factors and the time to complete each of them will be variable from patient to patient and between different treatment centers. For example, such factors may include resource availability (e.g. trained medical staff and equipment) as well as processing times required by CT scan equipment and other ancillary hardware and software to present data to physicians.

For the purposes of illustration, these factors are described in terms of a representative diagnosis and treatment scenario of a patient exhibiting symptoms of a stroke, the patient arriving at the emergency room of a treatment center and who thereafter receives the above CT procedures as part of the diagnostic protocol. Table 3 summarizes a number of the key process steps and typical times that may be required to complete each step.

Upon arrival at the treatment center, an emergency room physician conducts a preliminary assessment of the patient. If the preliminary assessment concludes a potential stroke, the patient is prepared for a CT scan. The time taken to initially assess a potential stroke patient upon arrival at the treatment facility may be 3-5 minutes.

Preparing the patient for a CT scan involves a number of steps including transferring the patient to the CT imaging suite and connecting an intra-venous line to the patient to enable the injection of contrast agent into the patient during the various CT procedures.

The CT scan includes conducting an x-ray scan of the patient together with a computerized analysis of the x-ray data collected. More specifically, as is known, during a CT scan, beams of x-rays are emitted from a rotating device through the area of interest in the patient's body from several different angles to receivers located on the opposite sides of the body. The received data is used to create projection images, which are then assembled by computer into a two or a three-dimensional picture of the area being studied. More specifically, the computer receives the x-ray information and uses it to create multiple individual images or slices which are displayed to the physician for examination.

CT scans require that the patient hold still during the scan because significant movement of the patient will cause blurred images. This is sometimes difficult in stroke patients and hence sometimes head restraints are used to help the patient hold still. Complete scans take only a few minutes.

Upon completion of the initial CT scan including the post-processing time to assemble the images, the physician interprets the images to determine a) if a stroke has occurred and, b) if so, to determine if the stroke is hemorrhagic or ischemic. If the stroke is hemorrhagic, different procedures may be followed. It will typically take the physician in the order of 1-2 minutes from the time the images are available to make the determination that the stroke is hemorrhagic or ischemic.

If the stroke is ischemic, the decision may be made to conduct a CT angiogram (CTA).

CT angiography procedures generally require that contrast agents be introduced into the body before the scan is started. Contrast is used to highlight specific areas inside the body, in this case the blood vessels. In addition because of presence of contrast in the very small vessels of the brain, overall the brain looks brighter (has a higher Hounsfield value) also known as contrast enhancement. Contrast agents are iodine based compounds that inhibit the passage of x-rays through the tissue. As such, they can be effective in enhancing the distinction between tissues where the contrast agent is present compared to those tissues where it is not. The CT angiogram may require additional preparation time but will typically not require that the patient be moved. Generally, CT angiogram procedures involve the injection of a bolus of contrast through an IV line followed by the CT scan. A typical contrast bolus may be 70-100 ml injected at 5 ml/second. The volume and injection rate of contrast is determined by the procedure being followed and is generally injected in a minimally sufficient volume to be present in the tissues of interest at the time the CT scan is conducted. Over a relatively short time period, the contrast becomes diffused within the body thereby providing only a relatively short window of time to conduct a CT procedure.

The CT angiogram data is substantially greater than what is collected from a basic scan and like a basic CT scan must be subjected to post-processing to create the images. The post-processing time is typically in the range of 1-5 minutes.

After processing, the physician interprets the data and makes a decision regarding treatment. Generally, the physician is looking to determine a) where is the occlusion? b) what is the size of the core? and c) obtain a qualitative feel for penumbra and collaterals.

Ultimately, and based on these factors, the physician is looking to make a decision on what brain tissue is worth fighting for. In other words, based on the combination of all these factors, the physician is looking to decide either that very little or no penumbra can be saved, or alternatively that it appears that penumbra can be saved and it is worthwhile to do so.

The CT angiogram provides relatively little data about collaterals and perfusion to the ischemic tissue as it is only a picture of the brain at one instance in time. That is, as it takes time for contrast agent to flow through the brain tissues and such flow will be very dependent on the ability of vessels to carry the contrast agent, a single snapshot in time does not give the physician enough information to make a diagnostic and/or treatment decision. Hence, either a multiphase CTA (mCTA) or a CT perfusion (CTP) procedures may be undertaken to give the physician a more quantitative sense of brain perfusion. Like CT angiogram, CT perfusion procedures involve the injection of contrast agent into the patient. It should also be noted that some centers may choose to do a CT perfusion study before the CT angiogram because they feel that the contrast injection from the CT angiogram interferes with the quality of data of the CT perfusion. Multiphase CTA is described in U.S. patent application Ser. No. 14/425,763 (and resulting granted U.S. Pat. No. 9,324,143) which is hereby incorporated by reference.

Perfusion computed tomography (CTP) allows qualitative and quantitative evaluation of cerebral perfusion by generating maps of cerebral blood flow (CBF), cerebral blood volume (CBV), and mean transit time (MTT). The technique is based on the central volume principle (CBF=CBV/MTT) and requires the use of complex software employing complex deconvolution algorithms to produce perfusion maps. Other maps such as Tmax maps may also be created.

CTP studies are acquired with repeated imaging through the brain while the contrast is injected. The technique varies significantly from vendor to vendor and also from center to center and hence requires specialized training with the specific equipment at each center. CTP typically involves imaging of the brain over approximately 60-70 seconds (at 1-4 second intervals) in order to acquire multiple images. The technique is quite vulnerable to patient motion and also requires the patient to hold still for the period. Furthermore, CTP also involves substantial radiation exposure in the range of 5-10 mSv as the number of images taken over the time period is significant.

The procedure generates a large dataset that must then be transferred to a dedicated workstation for post-processing. This step may take over 10 minutes in order to produce separate maps of each of CBF, CBV, and MTT. The perfusion maps are typically color coded maps.

Importantly, the post-processing requires the use of specialized and very often proprietary software that must be run by trained individuals. Ultimately, the time taken to fully complete CTP acquisition and analysis is highly variable as the above factors including the vendor, the speed of data transfer, local expertise, the time of day the study is being undertaken (i.e. working hours vs. after hours) as well as other factors can all have an affect on the actual amount of time required to complete the study.

TABLE 3
Typical Diagnostic Steps and Completion Times

| | | Elapsed | | |
|---|---|---|---|---|
| Procedure | Time (minutes) | Total | | Comments |
| Initial Assessment | 3-5 | 3-5 | | |
| Transfer and | 20 | 23-25 | | |
| Preparation for CT Scan | | | | |
| CT Scan | 1 | 24-26 | | |
| CT Scan Interpretation and | 2-3 | 26-30 | | CT Angiogram Preparation |
| CT Angiogram Preparation | | | | may be concurrent with |
| | | | | CT Scan Interpretation |
| CT Angiogram Procedure | 1-3 | 27-33 | | |
| CT Angiogram Post Processing | 2 | 29-35 | | |
| CT Angiogram Interpretation | 4 (minimum) | 33-39 | | CT Perfusion Preparation |
| and CT Perfusion Preparation | | | | may be concurrent with |
| | | | | CT Scan Interpretation |
| CT Perfusion Procedure | 1 | 34-40 | | |
| CT Perfusion Post Processing | Variable | 44-60 | | Will depend on vendor |
| | 5-20 (minimum) | | | specifics |
| CT Perfusion Interpretation | Variable | 46-70 | | Will depend factors |
| | 2-10 (minimum) | | | including: time of day; |
| | | | | center; vendor equipment |
| | | | | etc. |
| Transferring to angio suite | 20 min | 66-90 | min | |
| Placing patient on angio table | 20 min | 86-110 | min | |
| and preparing surgical site | | | | |

Given the data regarding ‘time is brain’ there has been a need to improve efficiency and workflow within the hospital. Most importantly, there has been a need for improved systems for ‘one stop shopping’ wherein the imaging of the patient to:

a. rule out a bleed (i.e. a hemorrhagic stroke);

b. detect a proximal vessel occlusion; and

c. assess the viability of the affected brain tissue

is performed at one location and at substantially the same time.

While there is the possibility of doing it in a way of a combined CT scan and angiography suite (one such solution is called the Miyabi CT), these are rare, very expensive and quite space consuming.

Accordingly, there has been a need for the endovascular surgical procedure (endovascular thrombectomy) to be performed in the angiography suite.

Thus there has been a need of creating the necessary imaging tools in the angiography suite that can perform the relevant imaging non-invasively obviating the need for a CT scan thus saving valuable time.

A list of references is provided at the end of the description which provide additional background on the state of the art relating to the field of stroke diagnosis.

### SUMMARY OF THE INVENTION

In accordance with the invention, systems and methods for diagnosing strokes are described. The systems and methods described herein enable faster diagnoses and treatments of different types of strokes by providing a physician with effective and timely information.

According to a first aspect of the present disclosure there is provided a method of imaging the brain within a patient diagnosed as potentially suffering a stroke, the method for deriving information about blood flow within the brain the method comprising the steps of:
a) obtaining a non-contrast rotational angiography scan of the patient's brain in the absence of contrast;
b) injecting a bolus of contrast agent into the patient;
c) obtaining contrast rotational angiography scan of the patient's brain at a time period after the bolus has been injected.

It will be appreciated that all of the rotational angiography scans may be performed using the same rotational angiography scanner.

The non-contrast rotational angiography scan may be the first imaging scan of the patient's brain after a potential stroke. The rotational angiography scanner may be configured to enable subsequent imaging of endovascular procedures

This may provide a method to bypass traditional cross sectional imaging (CT or MRI) in the workup of acute stroke. This may enable more rapid decision-making by excluding hemorrhage, detection of large vessel occlusion and ruling out a large amount of dead tissue in the affected territory by using a method comprising of multiphase rotational angiography in the surgical suite using the same equipment where the surgical procedure (e.g. endovascular thrombectomy) can be performed if needed.

Rotational angiography is a medical imaging technique based on x-ray that is configured to acquire CT-like 3D volumes. It is typically used for diagnoses, during surgery or during a catheter intervention. Typically, a rotational angiography machine uses a C-Arm. A C-arm is a medical imaging device with a C-shaped arm used to connect the X-ray source and X-ray detector to one another. The C-Arm is rotated around the patient and acquires a series or set of x-ray images in a single sweep that are then reconstructed through software algorithms into a 3D image. Rotational angiography may encompass flat-panel volume CT (where the detector is a flat panel configured to simultaneously record a two-dimensional image); and cone-beam CT (computed tomography).

In order to acquire a 3D image with a C-Arm, the C-Arm is positioned at the body part in question so that this body part is in the isocenter between the x-ray tube and the detector. The C-Arm then rotates around that isocenter, the rotation being between 200° and 360° (depending on the equipment manufacturer). Such a rotation takes between 2 and 20 seconds (e.g. 8 seconds), during which a few hundred 2D images are acquired. A piece of software then performs a cone beam reconstruction. The resulting voxel data can then be viewed as a multiplanar reconstruction, i.e. by scrolling through the slices from three projection angles, or as a 3D volume, which can be rotated and zoomed. A voxel is a three-dimensional pixel with a size typically around 0.625 mm×0.5 mm×0.5 mm.

The first contrast rotational angiography scan may take place a specific time period after the injection of the bolus of contrast agent. The time period should be configured such that the scan corresponds to a time at which the contrast has travelled from the injection point, through the heart, and into the brain. This time period between the bolus injection and the first contrast scan may depend on the patient (e.g. patient size, blood pressure, vessel tortuosity (which itself may be a function of age and genetic factors), heart rate); and the features which are desired (e.g. to highlight arterial flow, venous flow or collateral flow).

In a rotational angiography scan, the contrast may be timed so that it will highlight either the arteries or veins (venogram) or collaterals of interest.

The timing of the contrast rotational angiography scan may be based on a bolus-tracking angiography.

There are a few different ways of timing the contrast agent. These include one or more of the following:
a. fixed predetermined time based on experience. For example, a physician or computer may be able to determine a imaging time of x seconds after contrast injection. The determination may be based on one or more of, for example, age, tortuosity visible on non-contrast scan, cardio-vascular output (blood pressure, heart rate). This generally works well but leads to variation based on cardiac output etc.
b. continuous monitoring of a structure such as the carotid arteries in the brain. When these are seen the scan can be manually or automatically triggered. This may have the advantage that there is less inter-patient variation. A possible downside is that by the time the contrast is seen it may be too late.
c. doing a test run with limited contrast and timing its appearance (thereby giving a contrast transit time); noting it down and then injecting the full does and triggering the acquisition based on the measured contrast transit time.

The first contrast rotational angiography scan may take place between 5 and 15 seconds after contrast bolus injection.

The contrast bolus may be an iodine-based contrast agent.

The method may comprise, at regular intervals, obtaining n rotational angiography scans of the patient's brain. The n rotational angiography scans may be initiated at regular intervals separated by a time period t.

The method may comprise displaying the obtained sets of rotational angiography images as a time-sequenced series of images.

The time intervals between successive sets of rotational angiography images is selected based on the anticipated flow rate of contrast agent through the patient.

The brain may be automatically divided up into a number of zones of interest by identifying pre-determined anatomical zones.

The method may further comprise the step of:
identifying collateral vessels facilitating collateral blood flow within the contrast rotational angiography scan based on: opacity of the vessels; position of the vessels; and the time delay between the bolus injection and the contrast rotational angiography scan.

The method may further comprise the step of superimposing data from the multiple rotational angiography scans and allocating colour based on delay of opacification.

The method may comprise evaluating the change in density at the level of each voxel of brain tissue and using the time-contrast curve to create maps which shows the tissue at risk due to the intracranial occlusion. The time-contrast curve may be evaluated based on inflow and outflow of contrast at the tissue level. The creation of this map may facilitate the detection of the intracranial occlusion (by looking at the ischemic region, one can determine the expected site of occlusion) thus reducing the likelihood of false diagnoses.

The different rotational angiography scans may correspond to different radiation exposures, and the method comprises using fixed elements (e.g. parts of the brain or skull which do not change from scan to scan) from the scan with the highest radiation dose to increase the signal to noise ratio of at least one scan with the lower radiation dose. In this way, the highest signal to noise ratio may be used to create and add to the SNR of the datasets with the lower radiation dose.

The method may comprise:
calculating the rate of collateral flow based on the opacity profile of one or more identified collateral vessels recorded over the time period of the contrast rotational angiography scan.

The method may comprise:
identifying collateral blood vessels by identifying adjacent voxels in three-dimensional space which have a x-ray opacity exceeding a predetermined threshold.

The method may comprise:
defining an axial axis of each identified collateral vessel.

The method may comprise:
calculating the contrast profile based on aggregating x-ray opacity values of voxels at different axial positions along the axial axis of each identified collateral vessel.

The method may comprise:
calculating the x-ray opacity profile within an identified vessel at a point in time.

The variable opacification of the collateral vessel (as the contrast comes into the collateral) may be used to calculate flow rate within the collateral.

The clearing of contrast within the collateral (due to unopacified blood without contrast following the opacified contrast-enhanced blood) may be used to calculate the flow rate within the collateral.

The change of density or opacity of each voxel of brain tissue is calculated and the rise and fall of the density or opacity (based on appearance and disappearance of contrast at the capillary level) is used to create maps which demonstrate the ischemic tissue.

The combination of calculated flow rate within the collaterals and the voxel based map of the ischemic tissue is used to calculate and differentiate between tissue at risk (tissue that would die if recanalization is not performed) vs. tissue that is already dead.

The method may comprise:
using a non-transitory computer readable medium encoded with instructions to analyse the contrast rotational angiography images according to the following:
   map a plurality of zones of interest;
   analyze each zone of interest to assign a collateral value to each zone of interest where an assigned collateral value represents relative viability of collaterals within that zone and where the assigned collateral value represents the total collateral blood flow into the zone;
   calculating a secondary score based on a cumulative total of values from the assigned collateral values.

The zones of interest and the secondary score may correspond to the ASPECTS protocol.

ASPECTS (Alberta stroke programme early CT score) is a 10-point quantitative topographic CT scan score used in patients with middle cerebral artery (MCA) stroke. Segmental assessment of the MCA vascular territory is made and 1 point is deducted from the initial score of 10 for every region or zone involved:
caudate
putamen
internal capsule
insular cortex
M1: “anterior MCA cortex,” corresponding to frontal operculum
M2: “MCA cortex lateral to insular ribbon” corresponding to anterior temporal lobe
M3: “posterior MCA cortex” corresponding to posterior temporal lobe
M4: “anterior MCA territory immediately superior to M1”
M5: “lateral MCA territory immediately superior to M2”
M6: “posterior MCA territory immediately superior to M3”

The method may comprise:
identifying multiple fixed points associated with the patient's head during each rotational angiography scan;
identifying and correcting for movement of these fixed points during the scan.

The multiple fixed points may comprise inherent features of the patients head (e.g. skull features, ears, eyes, nose and/or brain features).

The multiple fixed points may comprise external markers affixed to the patients head.

The method may comprise:
obtaining one of the rotational angiography scans at a higher x-ray intensity than at least one other rotational angiography scan; and
using data from the higher x-ray intensity scan to constrain post-processing of the at least one other rotational angiography scan.

According to a further aspect, there is provided a system for imaging the brain within a patient diagnosed as potentially suffering a stroke, the system comprising:

Rotational angiography scanner, wherein the scanner is configured to
a) obtain a non-contrast rotational angiography scan of the patient's brain in the absence of contrast; and
c) obtain contrast rotational angiography scan of the patient's brain at a time period after the bolus has been injected.

According to a further aspect, there is provided a computer program for imaging the brain within a patient diagnosed as potentially suffering a stroke, the computer program being configured, when run on a computer configured to control a rotational angiography scanner, to:
obtain a non-contrast rotational angiography scan of the patient's brain in the absence of contrast;
obtain contrast rotational angiography scan of the patient's brain at a time period after a bolus has been injected.

In various embodiments, the number of phases can be varied, but preferably n is 1-6. The time period, t, can also be varied and may be selected based on a number of factors including the anticipated flow rate of contrast agent through the patient. The time period, t, may also be selected based on an initial diagnosis of the patient having suffered an ischemic or hemorrhagic stroke. For example, if the patient is suspected as having suffered an ischemic stroke, t will typically be 6-18 seconds. If the patient is suspected as having suffered a hemorrhagic stroke the time period t, is preferably 10-40 seconds.

In another embodiment, the method further comprises the step of: enabling a user to mark at least one zone of interest within one phase of the images to create a marked zone of interest and wherein a marked zone of interest represents any one of or a combination of asymptomatic tissue or symptomatic tissue. In one embodiment, a corresponding zone of interest of a single image on an opposite side of the brain is automatically marked based on the area and location of the at least one marked zone of interest. In one embodiment, a corresponding zone of interest in another phase is automatically marked to create further marked zones of interest based on the area and location of each marked zone of interest.

In another embodiment, the method further comprises the step of: calculating a contrast density value within each marked zone of interest. In one embodiment, contrast density values for each marked zone of interest are tabulated within a database.

In another embodiment, the method further comprises the step of: calculating and displaying a contrast density trend value from P1 to Pn for corresponding zones of interest across P1 to Pn on a symptomatic side.

In another embodiment, the method further comprises the step of: calculating and displaying a contrast density trend value from P1 to Pn for corresponding zones of interest across P1 to Pn on an asymptomatic side.

In a still further embodiment, the method further comprises the step of: comparing the contrast density trend value against a database of trend values to ascertain a collateral value for the marked zones across all phases.

In another embodiment, the method further comprises the step of: calculating and displaying a color code on at least one phase of images based on the collateral value or creating a colour coded map by summating the data from all the phases.

In another embodiment, the method further comprises the step of: calculating and displaying a change in contrast density of the entire brain from P1 to Pn.

In a still further embodiment, the method further comprises the steps of: identifying and marking one or more occlusions in one or more images in one or phases of the CT images and marking a downstream area relative to each marked occlusion; and, calculating and displaying a rate of opacification of vessels in the downstream area beyond each marked occlusion.

In yet another embodiment, the method further comprises the steps of:

identifying and marking corresponding symptomatic and asymptomatic regions of the brain; and calculating, comparing and displaying contrast density trends from the marked symptomatic and asymptomatic regions of the brain.

In yet another embodiment, the method further comprises the steps of: identifying and marking the location of an occlusion; calculating the diameter of vessels distal to the occlusion; identifying corresponding vessels on the contralateral side; calculating the diameter of vessels on the contralateral side; and comparing and displaying the differences in vessel diameter for each side for each of P1 to Pn,

In one embodiment, a method of deriving information about the location and properties of a blood clot/thrombus is provided wherein the method further comprising the steps of: enabling a user to mark a proximal end position of a suspected blood clot within at least one image of at least one phase of images; enabling a user to mark a distal end position of a suspected blood clot within at least one image of a later phase of images; and calculating and displayed a blood clot length based on the proximal and distal positions.

In one embodiment, a method of deriving information about the location and properties of a blood clot is provided wherein the method further comprises the steps of: enabling a user to mark a proximal end area of a suspected blood clot/thrombus within at least one image of at least one phase of images; enabling a user to mark a distal end area of a suspected blood clot/thrombus within at least one image of a later phase of images; calculating and displayed a blood clot/thrombus volume based on the proximal and distal end areas.

In another embodiment, the method includes the step of calculating a rate of change of contrast density within an intravascular blood clot/thrombus volume across different phases and correlating the rate of change to a known rate of change of contrast density within a blood clot/thrombus volume to determine a blood clot/thrombus permeability.

In another embodiment, the method includes the step of calculating a rate of change of contrast density within a blood clot/thrombus volume across different phases to a known rate of change of contrast density within a blood clot/thrombus volume to determine a blood clot/thrombus porosity.

Corresponding computer programs are disclosed. Computer programs may be stored in non-transitory media such as CDs or DVDs.

The methods disclosed herein may be implemented using a suitably configured computer processor in conjunction with memory (e.g. for storing a computer program and generated data) configured to control the rotational angiography machine and/or to process data received from a suitably configured rotational angiography machine.

### BRIEF DESCRIPTION OF THE DRAWINGS

The invention is described with reference to the accompanying figures in which:

FIG. 1 is a schematic diagram showing the relative effect of the time of a treatment decision to the benefit of a potential treatment with consideration to relative size of an infarct.

FIG. 2 is a graph showing time to re-perfusion and good clinical outcome.

FIGS. 3a and 3b are schematic diagrams showing the different methods for obtaining a MDCT scan and a rotational angiography scan.

FIGS. 4a and 4b are images of a flat detector rotational angiography scan and a multidetector computed tomography scan respectively.

FIGS. 5a and 5b are images of a flat detector rotational angiography scan and a multidetector computed tomography scan respectively.

FIGS. 6a and 6b are images of a flat detector rotational angiography scan and a multidetector computed tomography scan respectively.

FIG. 7 show illustrative scans where 3 sets (phases) of image data obtained on a CT scanner over approximately 8 second intervals through the entire brain of the patient; the first row (P1) being first phase data; the middle row (P2) being second phase data and the third row (P3) being third phase data.

FIG. 8 is a flow-chart showing the steps in creation of a semi-quantitative collateral map in accordance with one embodiment of the invention.

FIG. 9a is a contrast x-ray attenuation scan showing the flow of collaterals.

FIG. 9b is a schematic of how a contrast vessel axis is assigned.

FIG. 9c is a schematic of how opacity values are assigned to three-dimensional voxels.

FIG. 10 is a graph showing how the integrated measured intensity along a collateral vessel includes information about the contrast profile and the contrast flow rate.

FIG. 11 is a flow chart indicating how an ASPECTS score bac ne assigned to a brain based on determined collateral blood flow and tissue opacity.

FIGS. 12a-c are schematic diagrams of a rotational angiography scan indicating how motion correction may be performed.

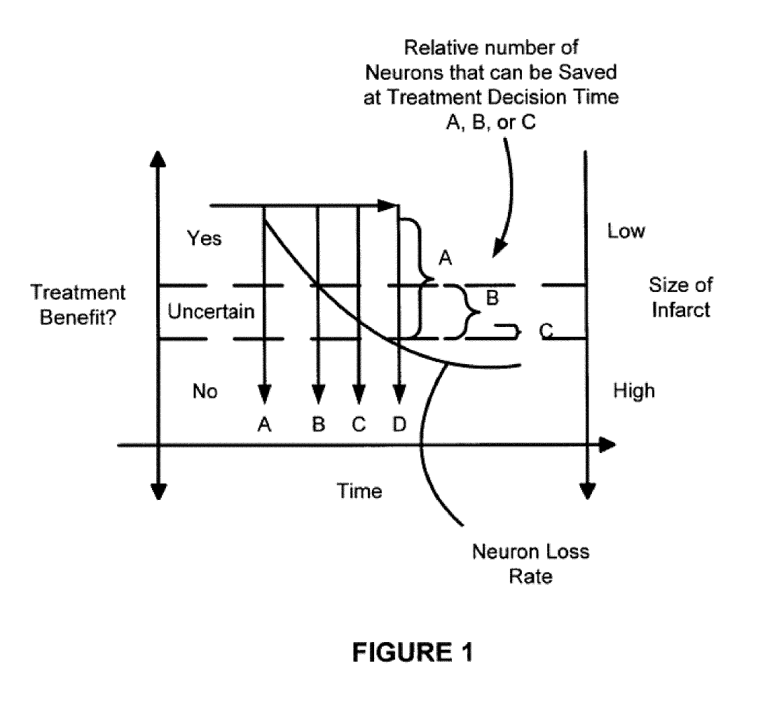

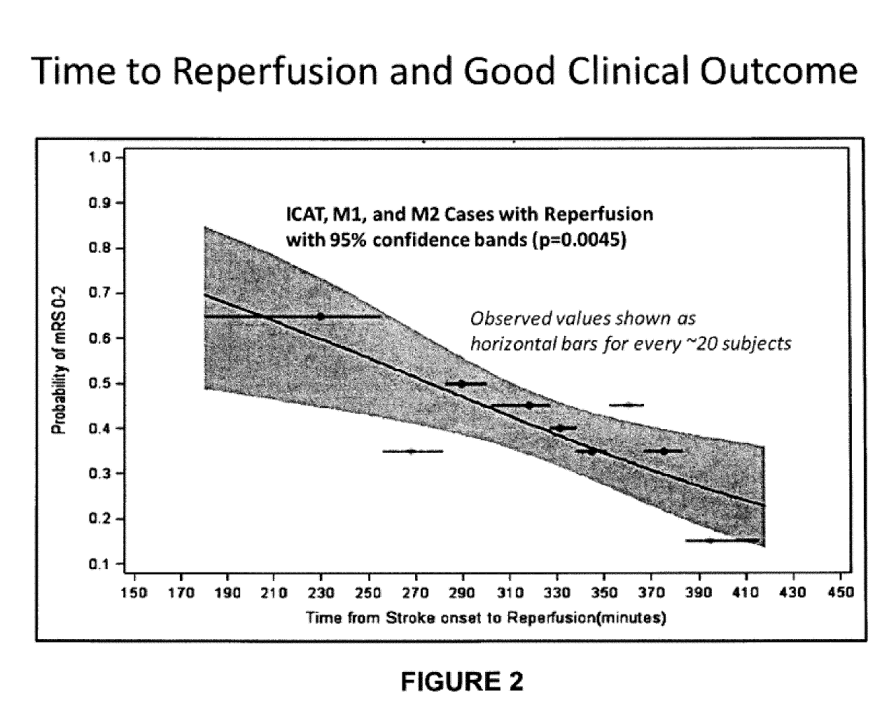

### DETAILED DESCRIPTION OF THE INVENTION

With reference to the figures, systems and methods for diagnosing strokes are described. More specifically, rotational angiogram techniques are described that can significantly improve the time required to effect an accurate diagnosis for a stroke patient by allowing bypass of the CT scanner. In addition, in many jurisdictions, patients are transferred from another hospital. In this situation, the patient has often already undergone a CT scan (which confirms the diagnosis of an ischemic stroke). However, given the time gap due to transfer time, there can be significant time savings by bypassing the CT scanner and getting all the relevant information from rotational angiography. Importantly, the procedures described herein allow for faster diagnosis of the location and extent of blockages as well as faster and semi-quantitative determination of the extent of the collaterals which will aid the physician in determining the treatment protocol.

In a first aspect, the invention involves conducting multiple rotational angiograms over a condensed period of time and at defined intervals. In a second aspect and from the image information obtained, the location and diameter of collaterals, the density of contrast and variance in the rate of filling of the collaterals is assessed in both space and time which is used to create a collateral map or collateral score. The collateral map or collateral score can be used by the physician to make a diagnostic and/or treatment decision.

Generally, in the context of this invention, and as explained in greater detail below, a collateral map is a visual representation of one or more time varied images of a section of the brain that show the collateral flow within regions of the brain. A collateral score is a grading system that represents the relative “strength” of collaterals.

In accordance with the first aspect of the invention, rotational angiography is a multiple image procedure conducted with a single bolus of contrast. It is typically conducted as 1-5 phases of rotational angiography at a 6-12 second (preferably about 8 seconds but could be up to 20 seconds or more) interval between the start of each rotational angiography sweep; however, the time interval may be longer in some circumstances. For example, during the work up of older patients or in patients with atrial fibrillation resulting in poor cardiac output, may suggest a greater interval. In addition, the time period may be varied between each rotational angiography sweep. The multiphase rotational angiography procedure produces a series of time-sequenced or phases of scans of the brain that provide information about the flow of contrast through areas of the brain from which the quality of perfusion and the quality of collaterals can be assessed and/or calculated. In some embodiments, the flow of collaterals can be inferred using a single rotational angiography scan due to the time period to complete the sweep of the head.

Below, after a comparison between rotational angiography and multidetector computed tomography techniques, the rotational angiography methodology is described in comparison to past procedures by way of example for typical cases to illustrate the distinctions between past procedures and some of the treatment scenarios where rotational angiography can provide significant advantages over these procedures. These examples are representative of various diagnostic scenarios that may occur at a treatment center and are intended to illustrate various time situations that could occur in the treatment of typical patients. The numbers and times discussed are not intended to be limiting.

### Comparison Between Rotational Angiography and Conventional CT Scans

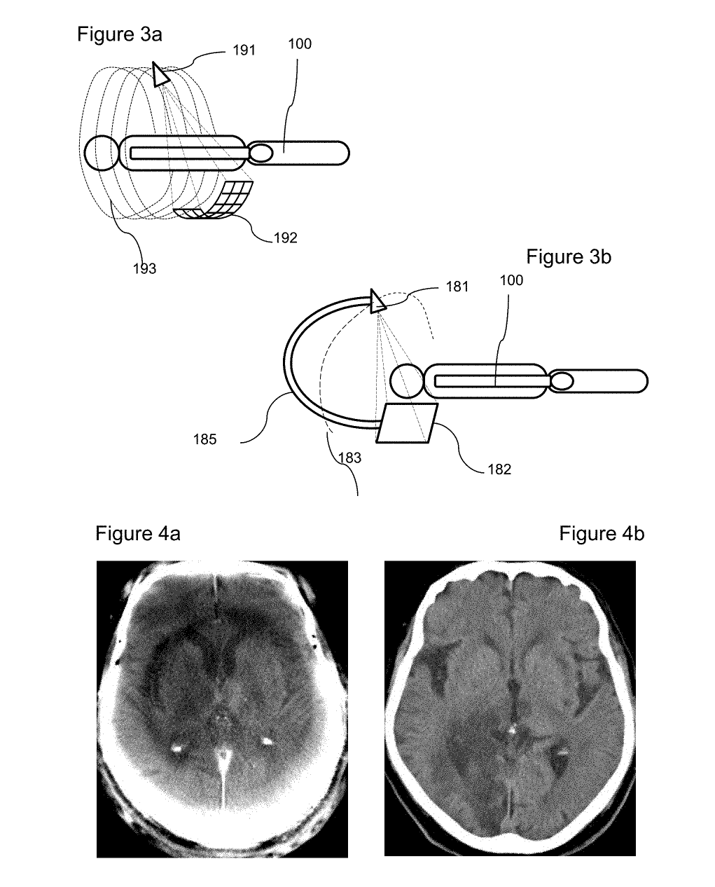

As shown in FIG. 3a, CT scans are now typically performed using Multidetector computed tomography (MDCT). This is a form of computed tomography (CT) technology for diagnostic imaging. In MDCT, a two-dimensional array of detector elements replaces the linear array of detector elements used in typical conventional and helical CT scanners. To perform a scan, the MDCT x-ray source 191 and detector 192 rotates in a helical path 193 around the patient 100 in order to complete the scan of an area of interest such as the head.

In contrast, in rotational angiography, as shown in FIG. 3b, the detector 183 is a large area detector, which is connected to the x-ray emitter 182 using a C-arm. In order to acquire a 3D image with C-Arm 185, the C-Arm is positioned at the body part in question so that this body part of the patient 100 is between the x-ray emitter 182 and the detector 183. The C-Arm 185 then rotates, the rotation 193 typically being between 200° and 360° (depending on the equipment manufacturer). Such a rotation takes between 2 and 20 seconds (e.g. 8 seconds), during which typically a few hundred 2D images are acquired. Software run on a computer processor then performs a cone beam reconstruction. The resulting voxel data can then be viewed as a multiplanar reconstruction, i.e. by scrolling through the slices from three projection angles, or as a 3D volume, which can be rotated and zoomed.

FIG. 4a shows an image of a brain carried out using a rotational angiography scanner which could be currently found in many hospitals. FIG. 4b shows the equivalent image carried out by using MDCT. With this technology, the images on rotational angiography scanner are significantly more difficult to interpret. Therefore, for example, although some features are visible, it may not be possible to accurately identify dead or dying brain regions. This means that, for example, it may be more difficult to score ASPECTS (a quantitative method of assessing the likelihood of recovery). This is a significant problem with the use of rotational angiography according to current methodologies.

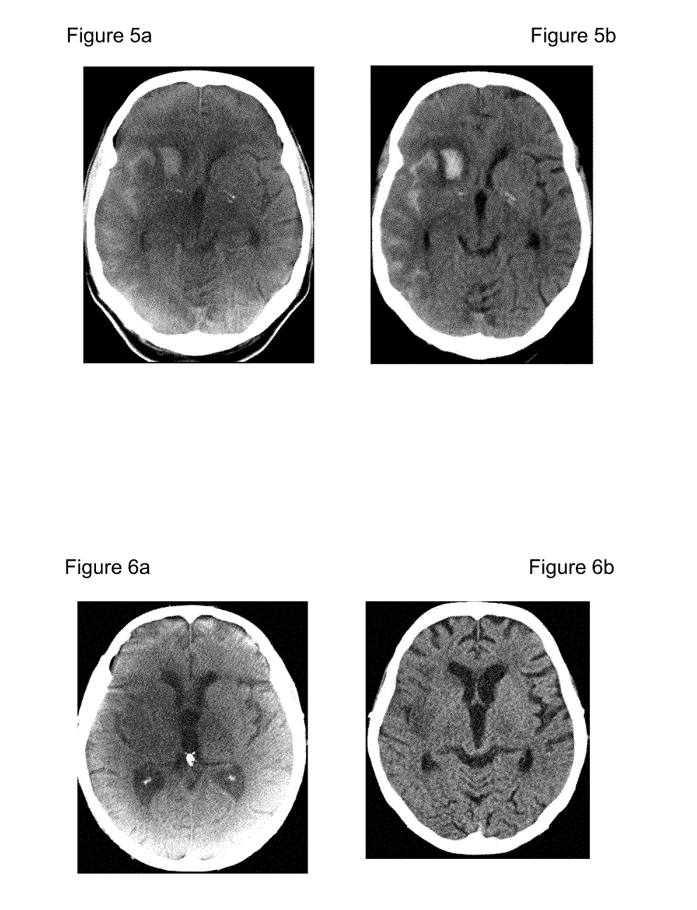

Nevertheless, the co-inventors have recognised that there are two aspects which may help overcome this issue. The first is that rotational angiography scanners are becoming more sensitive. FIGS. 5a and 5b show a comparison of a state-of-the-art (Siemens Artis Q) rotational angiography scanner and a MDCT scanner respectively of a brain with a bleed in the front left. A bleed is identified by a localised high-level of x-ray opacity corresponding to the blood absorbing the x-rays. In both the rotational angiography and MDCT scans, this feature is clearly visible.

FIGS. 6a and 6b show a further comparison of a rotational angiography and a MDCT scan respectively of a different brain. In both cases, the structure of the lobes can be clearly seen. As these images indicate, the capabilities of the rotational angiography scanners are approaching that of the MDCT scanners. However, there are significant limitations in the detailed reading of the brain imaging on rotational angiography. Generally, one of the current standards of quantifying the early changes in acute ischemic stroke is the ASPECTS scoring system. This is based on determining the absence of gray-white differentiation on the CT scan. This requires a certain quality of the CT scan images (as seen on the image produced using the routine CT scan). However the current quality of the imaging produced by rotational angiography does not allow for a good enough quality (as shown in FIG. 4a). Thus it is currently difficult to do conventional ASEPCTS scoring on the images produced using rotational angiography.

However, in addition to improving scanner technology, the inventors have realised that by detecting and monitoring collaterals within the brain, it is possible to determine which regions of the brain are receiving blood and therefore to determine or refine the ASPECTS score indirectly from collaterals rather than just directly making measurements of dead or dying brain based on regions of colour intensity from the x-ray images.

That is, in the case of strokes, the conventional blood path to areas of the brain is blocked. Without oxygen, those parts of the brain would die. However, collateral blood flow from other areas of the brain to these affected areas may provide sufficient oxygen to keep the affected areas alive or at least slow their deterioration. Therefore, the presence and extent of collateral blood flow is a good proxy measurement for inferring or deducing the ASPECTS score. That is, a determination of collateral flow can be used to help assign an ASPECTS score and/or identify the likelihood of the patient making a recovery with, for example, thrombolytic treatment.

### Representative Case Studies

### Scenario A: CT, CTA, CTP Procedure

A 72 year old man presents to the ER at 0820 hours. On examination, he has right hemiplegia and aphasia with an NIHSS of 19. As known, NIHSS is a stroke scale where the NIHSS number is derived from an examination of the patient. The scale range is from zero to 42 with 42 indicating that the patient is dead. Generally, a score of 10 or larger usually means a large stroke.

A quick examination of the patient is performed (5 min to complete). An IV line is started and blood is withdrawn for testing.

In the past, typically at this stage, the patient is transferred to a CT scanner (patient on the CT table at 0840 hours) where a non-contrast CT scan is performed. Subsequently after ruling out a hemorrhage a decision would be made to do a CTA and CTP. These would be acquired over the next 15 minutes. The images of the CTP would be transferred to dedicated software for post processing. The CTP maps, images of the CTA are reviewed at 0905 and a decision for treatment is made.

The patient would then be taken out of the CT scanner and transferred to the angio suite and reaches the angio suite at 0925 hours.

The patient is transferred to the angio table and made ready for the surgical procedure. The patient is ready for the surgical procedure at 0945 hours (85 minutes after getting to the hospital.

### Scenario B-Rotational Angiography

As in Scenario A, a 72 year old man presents to the ER at 0820 hours. On examination, he has right hemiplegia and aphasia with an NIHSS of 19.

A quick examination of the patient is performed (5 min to complete). An IV line is started and blood is withdrawn for testing.

Given the patient's symptoms the team decides that this could be a patient suitable for endovascular thrombectomy.

The patient is straight transferred to the angio suite and reaches there at 0840 hours.

A quick (non-contrast) Flat Detector Rotational Angiography is performed. This takes 2 minutes to perform and another 3 minutes of post processing. The team is able to rule out a hemorrhage.

While the nurse is preparing the surgical site, the team sets up for a multiphase FDCT (Flat-detector Computed Tomography also known as Rotational Angiography) which can be performed using the same equipment as that used to rule out a hemorrhage (this means that the patient does not need to be moved). This takes another 2 minutes of data acquisition. This allows the medical team to determine:
a. Evidence of a proximal vessel occlusion suitable for endovascular thrombectomy b. Evaluation of collateral maps using rate and extent of collateral filling.
This can be assessed regionally to correlate and determine the ASPECTS score and hence, determine viability of the brain tissue.

All these are completed in 10 minutes; in parallel the nurse has also prepared the surgical site.

Vascular access is obtained at 0850 (nearly one hour faster than the current traditional approach).

There are a number of reasons why the current workflow is performed in this way. In particular, CT scans have been the go-to scanning technology for many years. They are typically more available than rotational angiography machines as they require fewer specialized staff and they are often set up to be used at any time of the day or night. In contrast, a rotational angiography requires specialized staff and so may not be available 24 hours a day.

However in the recent past (as a consequence of the work done by the inventor; references listed below) endovascular thrombectomy is now the standard of care. As such there has been a dramatic increase in the number of procedures being performed and in many high-volume centres there is now the critical number of patients to create the necessary infrastructure to allow 24/7 access to the stroke team and equipment. Also, as illustrated above, the suggested workflow has the potential to dramatically improve workflow and hence improve patient outcomes.

Another important point is the ‘shelf life’ of the imaging. The brain cells are clearly dying during the early phase of an acute ischemic stroke and things are rapidly evolving. The only currently known way to stop this process is to establish reperfusion through the occluded vessel as quickly as possible. Thus as the time interval between the imaging (on the CT scan) and reperfusion increases the validity of the imaging data reduces. Overall this means that there will be a ceiling on the specificity and accuracy of the determination of ‘core’ or ‘dead brain’ on the CT scan as usually there is a time gap of 60-120 minutes between the CT scan and opening of the vessel.

Techniques have been described that are being used to predict the rate of brain infarction (by the inventor and his colleagues) using CT perfusion. However while these techniques can describe the rate of brain death and with further refinements can potentially predict this better, of course, these cannot change the course of brain death.

However, if a patient is in the angiography suite and the imaging is being performed using rotational angiography, the time interval between imaging and opening the occluded vessel is much shorter (10-40 minutes).

Thus, the likelihood of significant brain death in the interim is much lower. This also means that the need for the same degree of precision and predictive modeling is expected to be lower.

### Rotational Angiography Procedures and Interpretation

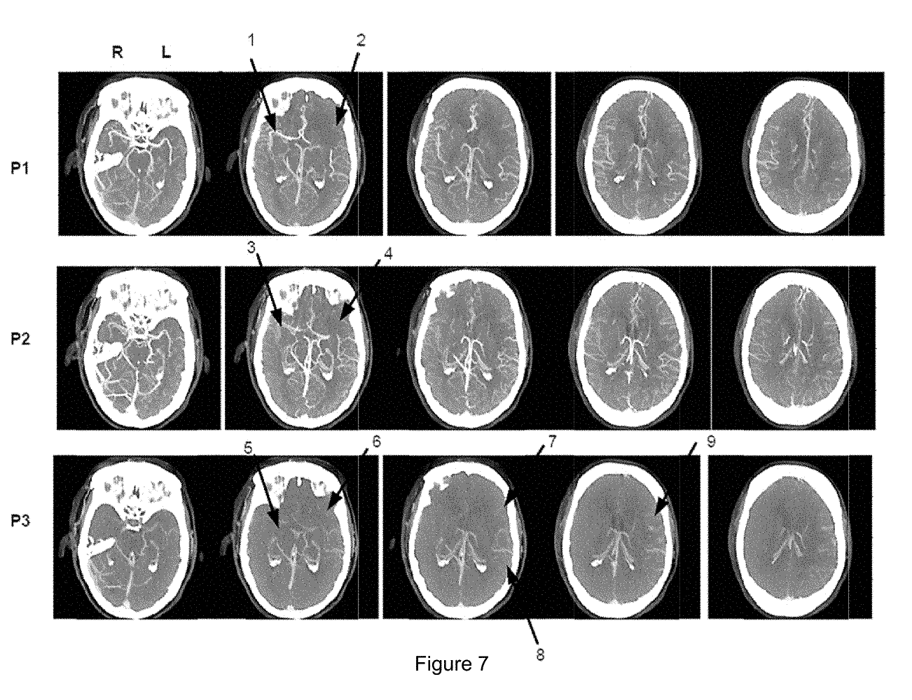

FIG. 7 shows a series of illustrative images which show how the data from rotational angiograms could be interpreted. In this case, the raw images are actually from a conventional CT scanner, but as slices from a CT scanner are qualitatively similar to slices from a rotational angiography scanner, we have used conventional CT scans to illustrate and compare how the interpretation of corresponding rotational angiography images would be the same or different from the interpretation of conventional CT scans.

More specifically, the first row of images in FIG. 7 shows 5 different spatial slices of a patient's brain at a first time, referenced herein as phase 1 or P1. The second and third row of images also show 5 corresponding spatial slices of a patient's brain at second and third times or P2 and P3 respectively at the same levels that the P1 images. In this case, each phase was initiated 8 seconds after the previous phase.

From the P1 images, it can be seen that the right-side vessels of the brain contralateral to the side causing the patient's symptoms, are unaffected as they can be seen as fully opacified (right middle cerebral artery branches) at P1 (arrow 1) whereas the left side (ipsilateral) is not opacified (arrow 2). In addition, it can be seen that posteriorly (PCA circulation), both sides are unaffected as the vessels are opacified. That is, the P1 scan shows that within a few seconds of injecting a contrast bolus, the contrast has effectively flowed to the anterior right side and the posterior regions of the brain and has otherwise been fully distributed as would be expected within healthy tissue. In comparison, at P1, arrow 2 shows that contrast has not fully perfused an area of the left side by the absence of a similar contrast density as compared to the right side. Thus, these P1 images are suggestive of a left side occlusion.

At P2, on the right side, contrast is passing through the contralateral vessels (arrow 3). Thus, the P2 images show a decreasing contrast density on the healthy right side. At P3, almost all of the contrast has passed and the contrast density is lower still on the right side (arrow 5).

At P2, on the anterior left side, the images show that some collaterals are filling due to an observed increase in contrast density at this level (arrow 4). At P3, the contrast density is increasing further (arrow 6). In addition, at other levels, a hold up of contrast can be seen in the left middle cerebral artery (MCA) region (arrow 7).

From these images, it is determined that the perinsular region (i.e. the region where the collaterals are weak (arrows 6, 7, 9)) is at a greater risk to die, whereas posteriorly (arrow 8), the brain may be salvageable.

Accordingly, from such time sequenced images, the physician has a basis on which to assess the quality of the collaterals. In this first example, collateral health is sufficiently robust to suggest potentially salvageable tissue and thus in conjunction with the patient's clinical symptoms may make the decision to conduct an intra-arterial recanalization treatment. It is noted that in corresponding rotational angiogram images, the opacity of volumes of the brain may be more difficult to ascertain. Nevertheless, the flow of contrast agent through collaterals may still be determined.

It should also be noted and as understood by those skilled in the art that the medical practitioner in making a diagnostic/treatment decision may also be making that decision based on a concurrent evaluation of the non-contrast scan (and other clinical data) which has already been performed and/or obtained from the patient.

In FIG. 7, the three rows represent the three phases P1, P2 and P3 with an approximate 8 second image interval (between the start of each sweep). In the P1 images, the arrow identifies an area with poor opacification in comparison to the posterior regions where there is strong contrast density. These images, when interpreted along with the non-contrast scan, also helps in a more accurate and precise determination of infarct core.

In the P3 images which are taken approximately in a sweep starting 16 seconds after the P1 phase started, the arrows show a hold up of contrast in the left MCA territory thus indicating that contrast is filling in through collaterals.

It is important to note that on the right side (normal side), the P3 images show near complete clearing of contrast from the arterial vasculature by the third phase which would be expected as contrast flows through unaffected vessels approximately 16 seconds after injection. This means that the direction of collateral flow can help indicate the location of an infarction as collaterals will generally flow into an affected area rather than out of a stroke-affected area (because conventional blood flow into the affected area has been restricted).

The images collectively indicate that the periinsular region (i.e. the area that shows poor collaterals) is at high risk to die; however further posteriorly and cranially, there are good collaterals likely representing salvageable brain regions.

### Semi-Quantitative and Quantitative Assessment of Collateral Strength

Rotational angiography scanning methodology can allow the physician to effect a timely diagnosis of the nature of a stroke by determining the collateral blood flow to particular areas of the brain.

Collateral flow can be detected in a number of ways including detecting a change in the opacity of particular areas of the brain and by detecting actual flow of the contrast agent through blood vessels in the brain.

In another aspect of the invention, methods of providing a quantitative or semi-quantitative assessment of collateral strength are described that are built from the rotational angiography images.

In this case, if a number of contrast rotational angiography scans are performed (multiphase rotational angiography), these provide data that is sequenced in time. The image data can be interpreted based on different input functions including:
a. Assessment of collaterals.
b. Assessment of contrast flow into particular non-affected regions (a proxy measurement of blood flow).
c. Assessment of collateral contrast flow into particular affected regions (a proxy measurement of blood flow).
d. Comparison of contrast density to the opposite side of the brain (e.g. not an absolute change in contrast density but a comparison to a corresponding area of the opposite side of the brain).
e. Change in contrast density of the entire brain over time.
f. Change in contrast density of vessels over time.
g. Rate of opacification of vessels beyond the occlusion.
h. Location of the occlusion. For example, for an M1 occlusion (proximal middle cerebral artery), collaterals come through leptomeningeal connections from the anterior cerebral artery and posterior cerebral artery while for an M2 occlusion (first order branch of the middle cerebral artery) collaterals come from the other M2 branch.
i. Diameter of vessels distal to the occlusion compared to the contralateral side.
j. Understanding the information on the multiphase CTA taking into account the patient's clinical information e.g. a patient with minor stroke symptoms with an MCA occlusion likely has excellent collaterals.

The presence of the collaterals can be used visually, semi-quantatively or be used to generate colour-coded collaterals maps to aid in decision making. It is expected that a very experienced user may be able to use visual cues just by looking at the collaterals to allow for fast decision making (similar to the use of mCTA) but a newer user would benefit from the more detailed an intuitive colour coded maps.

This colour coded maps would use information from
a. Degree of filling of collaterals
b. Rate of filling of collaterals across different phases
c. In addition (since unlike a CT scan) the acquisition of a single dataset of rotational angiography takes usually around 8 seconds; it is expected that during this time of data acquisition, collaterals are substantially filling in and the base data is changing during the acquisition itself.
d. This can be used to advantage. It is expected that if the direction of flow is known (which is known in this case based on known human vascular anatomy and the site of occlusion), one can utilize the varying density of a particular vessel over the 8 seconds of acquisition as the contrast fills in.
e. This is unique information that is currently not generally utilized.

The creation of collaterals maps can in various embodiments take combinations of these input functions into account.

For example, in one example, image data is processed to determine the flow rate of contrast agent through the brain. That is, by subtracting the non-contrast scan, the changes in opacity of the contrast carrying vessels can indicate the presence or absence (and quality) of collaterals. By identifying collaterals and quantifying the blood flow that they are carrying can provide a quantitative measure of the normalcy of circulation (or not). In other embodiments, the flow rate may be determined by determining how the position of the contrast within vessels changes across different contrast rotational angiography scans. That is the change in position divided by the time between scans will give an indication of the velocity.

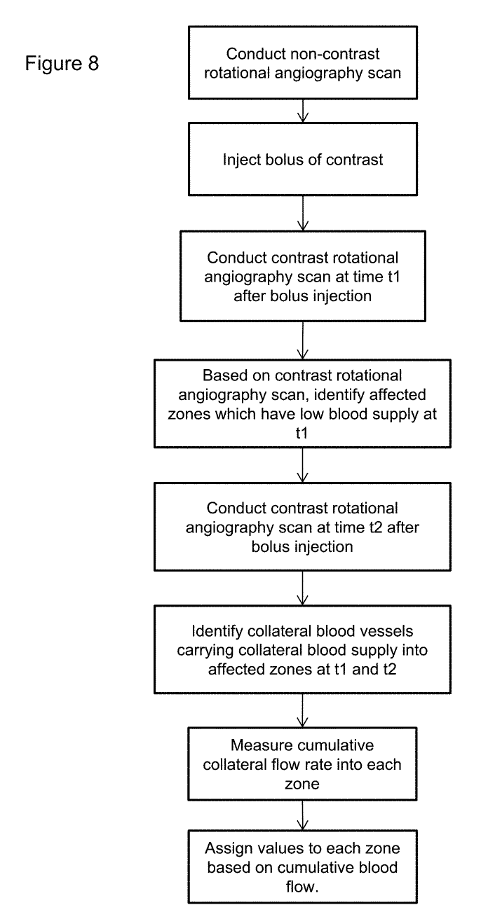

As shown in FIG. 8, a representative algorithm is described that can be used to provide a semi-quantitative assessment of collateral strength from the multiphase rotational angiography images. For each of the images from each of the phases, blood flow can be quantified for assisting in making a semi-quantitative assessment of collateral strength.

In one embodiment, multiphase rotational angiography software displays the multiphase rotational angiography images to the physician. For the P1 images, the physician is prompted to mark zones of interest including contralateral (asymptomatic) and ipsilateral (symptomatic) regions. In other embodiments, zones are automatically assigned by the system (e.g. to correspond to particular areas of the brain).

In healthy tissue, it would normally be expected that the degree of opacification would decrease from P1-Pn as contrast is passing through the vessels for the typical contrast injection volume and the time period between each phase. Thus, a rate of decrease in contrast can be calculated to provide a determination of the behavior of healthy tissue. In one embodiment, this comparison can be compared against typical or known rates of contrast as may be stored in a database.

In addition or alternatively, actual collateral contrast flow can be identified across the contrast scan phases based on position (collateral flow generally occurs in well known pathways), and time (because collateral flow is indirect, collateral contrast flow will typically be detected later than conventional healthy contrast flow).

In multiphase rotational angiography, the collateral flow can be identified by comparing multiple time-separate contrast scans. From these scans, the rate of flow of contrast can be calculated to determine the rate of collateral blood flow. From this, the rate of total blood flow to an ipsilateral region can be quantified to determine whether this region has a good change of recovering.

Color coding of the rate of change of contrast density and/or marking the collateral vessels themselves may be used to provide the physician with a readily identifiable visual indicator of the relative tissue health around the collaterals. For example, the contralateral region (or the collateral vessels) may be marked with shades of red indicating healthy perfusion. The ipsilateral region may be marked with color shades ranging from blue (indicating ischemic tissue) to red or green (indicating healthy tissue).

Further details of a methodology of assessing collaterals is described by the present technique and specifically the technique being used to identify retrograde filling pial arteries in the MCA territory distal to the occlusion. Pial arteries are distinguished from veins based on morphology, direction of filling and whether visualized early or late. These retrograde filling pial arteries are divided into 2 groups based on origin from anterior or posterior circulation; namely Anterior cerebral artery (ACA) to MCA and Posterior cerebral artery (PCA) to MCA and assessed for the following 2 properties using a grading system:
a) Prominence of pial arteries when compared to similar vessels in the opposite MCA territory (Same or more prominent=2, thin=1, minimal or not visualized=0) on any of the phases.
b) Rate of retrograde filling from parasagittal region to the sylvian sulcus. (Sylvian sulcus filling in first phase=2, in second phase=1, in third phase or not at all=0).

In case of a proximal M2 MCA segment occlusion, the same scoring template is used either in the anterior or in the posterior MCA regions depending on whether a dominant anterior or posterior M2 segment is occluded.

A scoring template as above results in a 4 point score for collateral assessment in the anterior and posterior MCA regions individually. A total score of 0-1 will be considered poor collateral status, 2 will be considered moderate and 3 good and 4 excellent collateral status for M2 MCA +/− intracranial ICA occlusions. A score of 0-2 will be considered poor collateral status, 3-4 will be considered moderate and 5-6 good collateral and 7-8 excellent status for patients with M1 MCA +/− intracranial ICA occlusions. For imaging selection, recanalization in any patient with poor collateral status in either anterior or posterior MCA regions (score 0-1) is likely futile.

Image quality may also be assessed. A good first phase is when convexity pial arteries are well seen on the contralateral asymptomatic hemisphere. If patient factors like congestive cardiac failure, atrial fibrillation, hypotension or contralateral proximal ICA stenosis or technical factors like early triggering of scan acquisition relative to contrast bolus injection limit visualization of convexity pial arteries in the first phase on the contralateral asymptomatic hemisphere, then this scan is considered sub-optimal. However, collateral assessments may still be carried out if the third phase on the contralateral asymptomatic hemisphere is in the late venous phase. If not, this scan cannot be used for collateral assessment. One easy solution for this is to add additional phases.

When an area has a poor collateral score as discussed above, this will mean either the tissue is already dead or the tissue is about to die and would be dead by the time the vessel can be opened making it a case of futile recanalization.

However given the overall dramatic improvement in workflow and reduction in the time gap between collateral imaging and opening of the vessel it is expected that the definition of poor collateral flow may evolve over a period of time. As explained above, in the case of CT scan, a patient with poor collaterals is still usually 90 minutes away from the opening of the blood vessel and restoration of blood flow; however, since the patient is already on the angio table, in this case the patient may be only 10 minutes away from the opening of the blood vessel. Thus while using the traditional approach, the patient may have a bad outcome because the brain continued to die for the next 90 minutes due to poor collateral flow, in the currently suggested approach, there may not be sufficient opportunity for the brain to die. Thus it is likely that the definition of poor collateral flow may be different in CTA vs. using multiphase rotational angiography.

The hardware and software to enable multiphase rotational angiography requires modification of known rotational angiography imaging equipment to enable the display of the images to the physician (and/or technicians) and to enable practitioners to input appropriate markings to the images for subsequent calculations. That is, the system provides appropriate computer input systems for point, line or shape marking for the purposes of identifying and/or delineating points, areas or zones of interest. Appropriate scales are supported to enable consistent comparison between marked areas on an images and comparisons across patients. Back end computer systems, user interfaces and network configurations enable the effective support for the various computational algorithms and the sharing or distribution of data across both local and wide area networks.

Determining Collateral Flow Rates from a Single Scan

Unlike conventional CT scanners, the single rotating sweep of a rotational angiography scanner typically takes several seconds during which time data corresponding to the entire region of interest is being recorded. Typical scan rates roughly in the range of between 25°/sec. and 55°/sec. Some scanners may rotate at a rate of up to 80-90°/sec and this may become more rapid as technology develops. This means that the contrast will change position within the patient's brain as the scanner is performing its sweep. In major vessels, the flow rate of blood can be in the region of 70-100 cm/sec. In smaller vessels that are backfilling through collaterals, the flow rate may be smaller (e.g. 1-2 cm/sec).

This will result in the measured integrated opacity of the collateral vessels being a function of the flow through those vessels during the time of the scan. That is, a volume of contrast agent will contribute to opacity at one position when the scan is initiated and then contribute to opacity at a different position within the vessel by the time the scan ends due to the motion of the contrast agent within the vessel during the scan.

Because these data of the flow rate encode flow information in the single three-dimensional scan, some embodiments may be configured to determine the rate of flow through particular vessels from a single rotational angiography scan. The collateral blood flow may be considered to correspond to an integral of the contrast flow through the vessels over the time period of the scan.

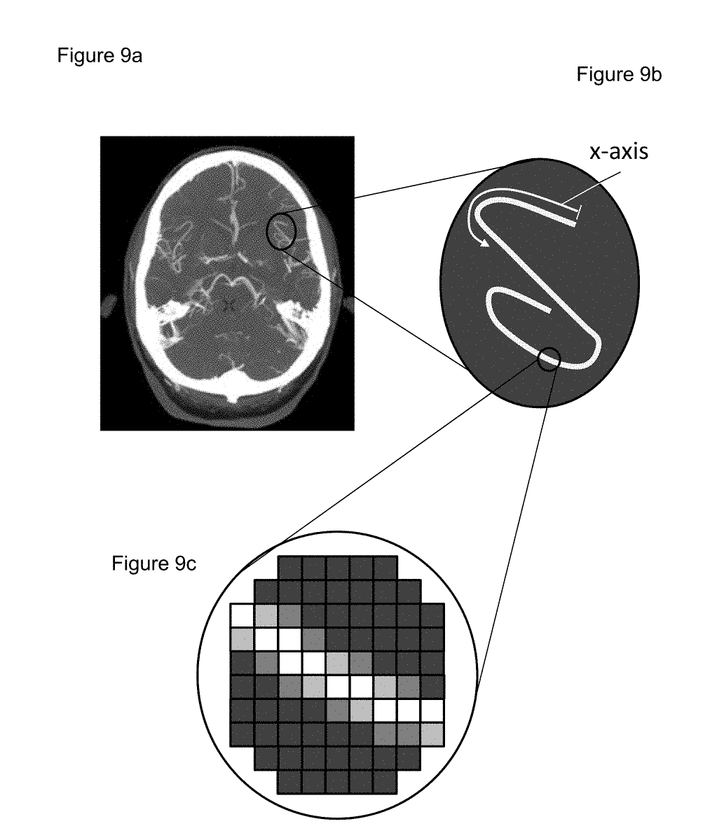

FIG. 9a-c shows an illustrative scan of contrast flowing through the patients brain 12 seconds after the arterial phase. The rotational angiogram may be configured to detect blood vessels which are carrying contrast agent (in these images, x-ray opaque regions are shown in light areas and high x-ray transparency corresponds to dark areas). During processing of the scan, the rotational angiogram is able to identify a portion of the vessel which is carrying contrast agent. This may be done by identifying adjacent voxels in three-dimensional space which have high opacity. A vessel which has been so identified is shown in expanded form in FIG. 9b. Based on the identified vessel, a curved vessel axis may be determined which lies along the core of the identified high opacity voxels (for clarity, in FIG. 9b, the vessel x-axis is shown offset from the centre of the vessel).

In FIG. 9c, a close up of the scanned voxels corresponding to a portion of the identified vessel is shown. The squares are 2D pixels which correspond to 3D voxels in three-dimensional space. It will be appreciated that where a voxel is entirely within the identified vessel, the opacity value (indicated by the lightness or darkness of the square) associated with that voxel will be dependent on the concentration of contrast within the vessel. Likewise, the opacity value associated with voxels outside the identified vessel will typically be darker as they are not affected by the contrast within the vessel. Some voxels may span regions within the vessel and regions outside the vessel. These spanning voxels will have an intermediate opacity value which is only partially dependent on the concentration of the contrast within the vessel.

To provide an intensity profile for the rotational angiography scan, the processor, in this case, is configured to integrate (or add) the opacity values for voxels corresponding to particular positions on the vessel x-axis. In this case, for each x-position, the system is configured to add the voxels opacity values within a transverse range of the determined x-axis. The extent of this range may be predetermined and/or depend on the transverse voxel opacity profile (e.g. the system may be configured to not add voxels which are less than 1/10^{th} of the background corrected peak voxel opacity). The system may be configured take into account the background by subtracting a background opacity value from each counted voxel. The background opacity value may be determined empirically by averaging the opacity values of a brain region which is not part of an identified contrast vessel.

By determining integrated opacity at points along the identified contrast vessel, it is possible to determine the integrated opacity or intensity, Iₘₑₐₛ(x, t), of the contrast vessel as a function of position along the vessel x-axis. The integrated or measured contrast-agent function along a particular vessel will depend on axial position (i.e. at a moment in time, the contrast agent will have a particular concentration profile) and time (i.e. to take into account the motion of the contrast agent and optionally changes in contrast profile). It may be assumed that any variance in the detected signal due to the changing position of the vessel itself with respect to the moving source and detector will be accounted for in the normal post-processing of the data to generate the 3D image.

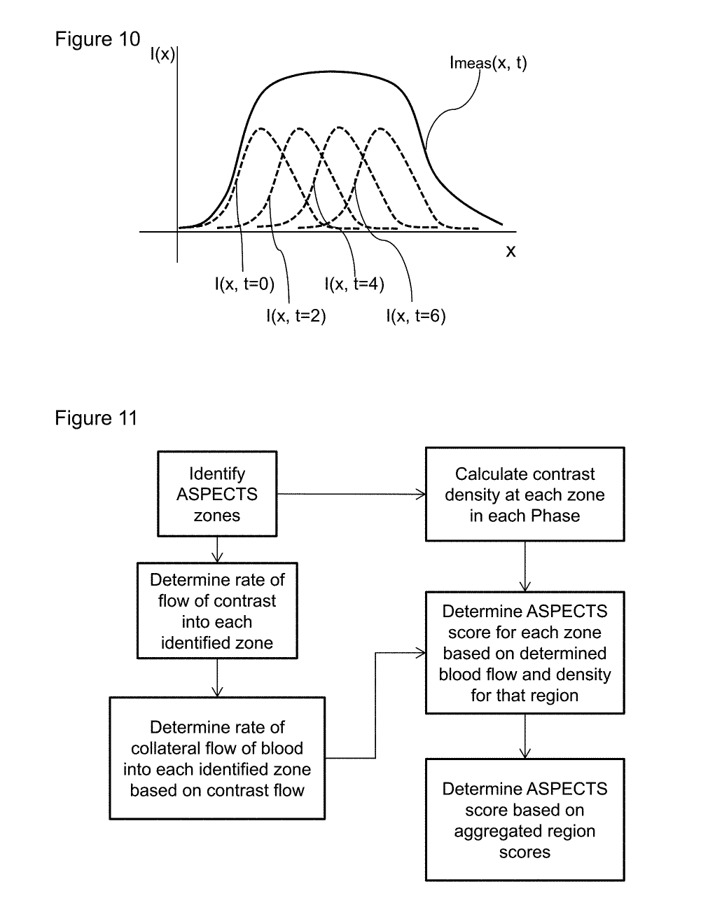

The measured intensity of the contrast along the axis of a vessel is shown in FIG. 10. Also shown in FIG. 10 are snapshots of the contrast profile are provided at 2 second intervals. These instantaneous profiles combine to build up to the integrated contrast intensity, Iₘₑₐₛ(x) which is a function of the position, x, along the collateral vessel axis.

That is, to determine the rate of flow through a particular vessel, the system may be configured to determine the integrated contrast opacity along a particular vessel. This can be performed by adding up the opacity values of voxels associated with a particular vessel (and optionally subtracting the corresponding background voxel opacity values of the corresponding non-contrast scan). This can be displayed as the quantity of contrast as a function of length along the vessel.

To determine the flow rate from the measured intensity profile along the vessel, it will generally be necessary to account for the instantaneous contrast profiles. One way of doing this would be to model the expected contrast profile based on, for example, one or more of quantity of contrast agent added, injection profile of contrast agent (e.g. was it injected evenly over a particular period of time), the distance and time from injection, and the size of the vessel concerned.

Another way of determining the contrast profile would be compare with other vessels of similar size within a non-stroke-affected region of the brain which have blood flow at the same time as the collateral blood flow. Using a comparative technique may better account for variables such as injection profile and distance and time from injection as these may be assumed to be largely the same for both vessels.

It is important to note, that this technique is intended to at least provide an indication of whether a particular brain region is salvageable in response to a particular treatment. Therefore, it is important that the results are reproducible within that context rather than deriving an absolute value of flow rates within vessels per se.

It will be appreciated that the above discussion is based on an effective constant radiation dose rate. Another way of gaining information on the instantaneous contrast rate at particular times would be to provide a time-varying dose rate. For example, if the dose rate was increased for a short period (e.g. 0.2 seconds) every two seconds, the integrated opacity of the contrast agent may show a series of peaks corresponding to the peak of the instantaneous profiles at these over-exposed times. That is, the intensity profile of the source may be accounted for in the post-processing for the stationary components of the region of interest, but moving or flowing portions may be distinguished. The spatial separation of these peaks would provide an indication of the flow rate.

### Aspects

As noted above, the determination of collateral flow (whether through multiphase rotational angiography or single scan rotational angiography) may help determine the ASPECTS score. ASPECTS (Alberta Stroke Program Early CT score) is a 10-point quantitative topographic CT scan score and was developed to offer the reliability and utility of a standard CT examination with a reproducible grading system to assess early ischemic changes on pretreatment CT studies in patients with acute ischemic stroke of the anterior circulation.

In the context of the present disclosure, rather than use CT scans, rotational angiography scans will be used to determine an equivalent measure which corresponds to the ASPECT score.

In conventional ASPECTS, 1 point is subtracted from 10 for any evidence of early ischemic change for each of the defined regions or zones. The defined regions consist of:
C—Caudate,
Insular ribbon,
IC—Internal Capsule,
L—Lentiform nucleus,
M1—Anterior MCA cortex,
M2—MCA cortex lateral to the insular ribbon,
M3—Posterior MCA cortex,
M4, M5, M6 are the anterior, lateral and posterior MCA territories immediately superior to M1, M2 and M3, rostral to basal ganglia.

Subcortical structures are allotted 3 points (C, L, and IC). MCA (middle cerebral artery) cortex is allotted 7 points (insular cortex, M1, M2, M3, M4, M5 and M6).

A normal CT scan receives ASPECTS of 10 points.

FIG. 11 shows a flow chart of an embodiment which is configured to determine an ASPECTS score for a brain based on a single contrast rotational angiography scan. In this case, the system is configured to identify the 10 ASPECTS regions from the rotational angiography scan. In other embodiments, this step may be performed manually (e.g. by a physician highlighting regions of interest) or other regions may be identified.

Once the ASPECTS regions have been identified, the system is configured to determine the opacity of the brain tissue within the identified zones. This opacity is an indicator of whether the brain within the regions is healthy, dead or dying. This part of the procedure is analogous to a physician manually calculating an ASPECTS score from a CT scan image.

In addition, the system is configured to determine the rate of flow of contrast into each region via collateral vessels. This can be determined as outlined above. From the rate of flow of contrast, a measure of collateral flow into the identified regions can be determined. The quantity of collateral flow provides an indication of how much blood flow is reaching particular regions despite the blockage.

The system is then configured to assign an ASPECTS score for each region based on the measured opacity (how healthy is the tissue in that region) and the determined blood flow (a measure of how quickly the tissue is likely to die).

It will be appreciated that in multiphase rotational angiography, the rate of change of opacity between multiple rotational angiography scans may also be used to inform how quickly the brain tissue is dying. In other embodiments, the ASPECTS score may be determined solely from a measure of collateral flow into the identified regions.

Using collateral flow determinations may be particularly used for ASPECTS zones M1 through M6.

There is also the potential in the future to modify the ASPECTS score or create an alternative score that is primarily based on the cortical regions. Also in the case of T occlusions (ICA+M1+A1) there will also be the opportunity to evaluate the collaterals in the ACA territory thus including this in the potential new scoring system.

### Dose Control

Because rotational angiography irradiates a large volume of tissue, the radiation risk may be more than would be the case for a targeted conventional CT scan. However, the aggregate dose across the multiple phases of rotational angiography scans may be reduced by utilizing data from previous scans to allow the dose administered in subsequent phases to be reduced.

For example, it could be assumed that aspects of the brain could be identified which would not change across the multiple scans (e.g. skull structure, structure of lobes). This means that one scan performed with a higher dose (and a lower signal to noise ratio) may be performed to determine the relative spatial location of these unchanging aspects of the resulting image.

Other images may be taken at lower dose (which a correspondingly higher signal to noise). However, by taking into account the known structures (or fixed elements) of the head (e.g. skull and brain) differences in these lower dose scans can be assigned to structures which may change during the multiphase rotational angiography scans. For example, changes may include regions of the brain changing opacity and particular vessels changing opacity due to the presence or absence of contrast agent. By using high-dose images to inform the processing of data from low-dose images, the data retrievable from the low-images may be kept high while lowering the aggregate dose over the entire procedure. That is, the signal to noise ratio of the lower dose scans can be enhanced.

This can be thought of as a type of data differencing. Data differencing consists of producing a difference given a source (the high-dose image) and a target (the low-dose image). In this case, the low-dose image contains the same amount of data (i.e. the location and intensity of each voxel). However, by constraining the intensity of particular voxels (e.g. by making skull voxels the same intensity as the high does image, and/or limiting the change in intensity for non-vessel voxels), changes in intensity between subsequent scans can be better assigned to the remaining voxels during post-processing (post-processing converts a time dependent 2-dimensional image or series of images into a three-dimensional static image).

### Motion Correction

Another issue with the longer timescale of scan associated with rotational angiography is patient movement. That is, if the patient moves their head during the scan, it is more difficult to recreate the three-dimensional axis based on the time dependent series of two-dimensional images obtained during the sweep. Patient movement may arise from a lack of control due to the stroke itself, involuntary movements or non-stroke-related medical problems.

One way of accounting for this motion is to determine multiple fixed points on the patient's body. If, for example, the patient is stationary for the first half of the scan and then moves their head rapidly at the midpoint, and then is stationary for the second half of the scan, it may be possible to identify the position of the reference points in the first half and the new position of the reference points in the second half of the scan. By applying a three-dimensional rotation to align the reference points, the data from the entire scan can be combined.

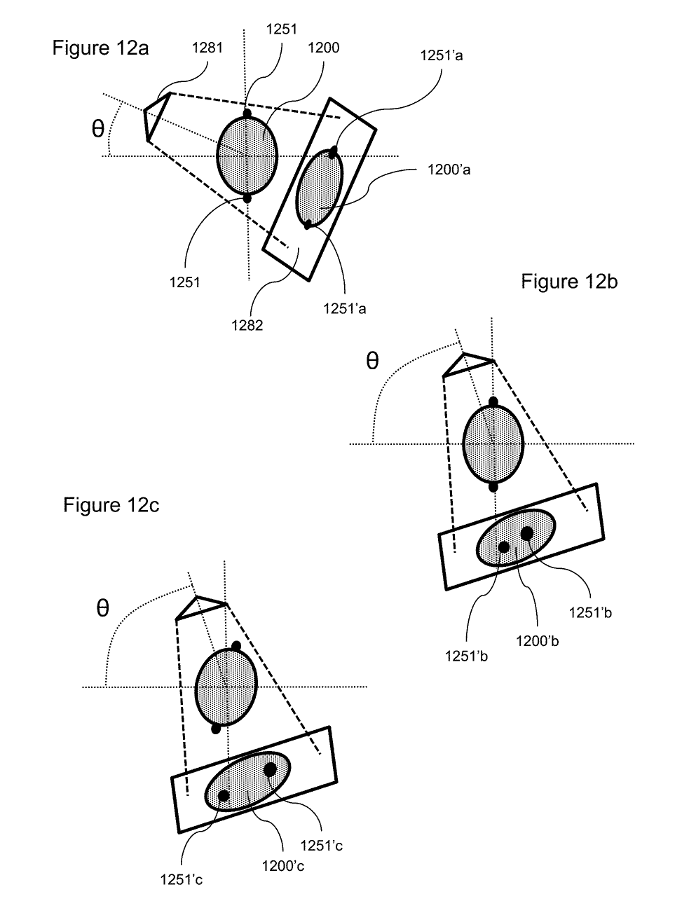

An example of this is shown in FIGS. 12a-c. These figures show a patient's head 1200 between the source 1281 and the detector 1282 of a rotational angiography scanner at two different times in a single sweep. The sweep corresponds to a single scan between θ=0 and θ=200 in this case. FIGS. 12a-b show the normal progress of the rotational angiography scan in which the C-arm (having source 1281 and detector 1282) rotates around a stationary patient. FIG. 12c shows the case in which the patient's head has moved. Therefore, the patient's head and the C-arm motion should be taken into account when reconstructing the scan.

Attached to the user's head are two reference point markers 1251, 1252. In this case, they are two solid objects with known size and with known x-ray attenuation. The position and angle of the head can be determined by mapping the position and size of the x-ray shadows of the two markers 1251′a-c, 1252′a-c on the two-dimensional x-ray screen 1282. That is, the angle of the person's head can be determined from the distance between the marker shadows 1251′a-c, 1252′a-c and the position of the marker shadows 1251′a-c, 1252′a-c because the shadows are a projection of the markers 3D position onto the 2D detector plane. As shown in FIGS. 12b and 12c, the relative and absolute positions of the markers shadows are different for the same θ due to a rotation of the patients head.

The size of the shadows may also be used to determine the location of the markers. That is, because the x-rays are divergent in the form of a cone, a marker will form a larger x-ray shadow when positioned closer to the source than when positioned farther away. This can be seen in FIG. 12b where the shadow 1251′b of marker 1251 is lamer than the shadow 1252 of marker 1252′a because marker 1251 is closer to the source of the diverging x-ray source than marker 1252.

The reference points may be inherent features of the patient (e.g. of the skull) which are identified automatically by the system or by the physician or medical personnel based on a previous scan data. Alternatively, as described above, the procedure may involve affixing markers to the user prior to scanning. These markers should have a particular x-ray attenuation and/or shape and/or size (so that they can be readily identified by the scanner) and be fixed relative to the patient's head (e.g. in the form of a head band). In some embodiments, the scanner may detect movement by detecting the relative movement of these markers which exceeds the expected relative movement due to rotation of the scanner and source.

It will be appreciated that this method is more viable in a rotational angiography scan than in a conventional CT perfusion scan, because in rotational angiography, the reference points will typically always be within the x-ray beam and in addition, the time of acquisition is much shorter (typically 8 seconds) compared to usually around 60 seconds in CT perfusion.

### Detection of Tandem or Multiple Occlusions

It is well known that in stroke patients more than one vessel may be occluded e.g. in a single patient the internal carotid artery and an M2 branch of the MCA may be occluded or as in another example the M1 segment of the MCA may be occluded along with the A2 segment of the anterior cerebral artery (ACA).

In such cases detection of the second occlusion especially in the setting of an emergency can be difficult and smaller occlusions may be overlooked.

However by visualizing the rate of filling and hold up of the affected territory (the region of the brain being supplied by the occluded vessel) through collaterals, one can deduce the presence of the second occlusion thus reducing the likelihood of an error.

### Discussion

Importantly, the rotational angiography techniques as described above provide numerous advantages over currently used CTA and CTP procedures in the diagnosis of ischemic stroke.

Importantly, the rotational angiography data that is collected over the typical 2-5 cycles provides the physician will a sequential series of data that can reveal changes in density within the collateral network over a known period of time.

### Intravascular Clot/Thrombus Identification and Quantification

In another aspect of the invention, blood clots causing an ischemic stroke and parameters describing the clot can be determined from appropriate graphical user interface and the addition of further processing algorithms as described below.

That is, in proximal artery occlusion it is helpful to the endovascular surgeon to understand more about the nature of the clot causing the stroke. In particular, it is useful to know the exact length of the clot and its relative permeability. These parameters can be difficult to determine using traditional CTA where only the proximal end of the clot can be identified. Moreover, this information cannot usually be obtained on the CTP images without a detailed study of the source images that be quite time consuming. The rotational angiography procedures allows for a quick determination of this length (and/or other dimensional parameters) which has implications in decision making such as choosing the length of the clot retrieving stents (e.g. stentriever length) at the time of the recanalization procedure.

In addition, the degree of porosity or permeability of the clot may have implications on the response to intravenous thrombolytic therapy.

Although the present invention has been described and illustrated with respect to preferred embodiments and preferred uses thereof, it is not to be so limited since modifications and changes can be made therein which are within the full, intended scope of the invention as understood by those skilled in the art.

### REFERENCES

“Time is brain-quantified”. Saver J L, Stroke. 2006, 37(1):263-6. Epub 2005.
“Evaluation of an Acute Stroke Patient with Flat Detector CT Prior to Mechanical Thrombectomy” Amelung et al., J Thrombo Cir 2016, 2:3.
“The Utility of DynaCT in Neuroendovascular Procedures”, Heran et al., AJNR 27, 2006.
“Analysis of Workflow and Time to Treatment on Thrombectomy Outcome in the ESCAPE Randomized Controlled Trial”, Menon et al. Circulation, 2016.
“Endovascular thrombectomy after large-vessel ischaemic stroke: a meta-analysis of individual patient data from five randomised trials”, Goyal et al, The Lancet, 2016.
“Time to Treatment With Endovascular Thrombectomy and Outcomes From Ischemic Stroke: A Meta-analysis”, Saver et al. JAMA. 2016
“Consistently Achieving Computed Tomography to Endovascular Recanalization <90 Minutes”, Goyal et al, Stroke, 2014.
“Clots, Collaterals, and the Intracranial Arterial Tree”, Menon and Goyal, Stroke, 2016.
“Analysis of Workflow and Time to Treatment and the Effects on Outcome in Endovascular Treatment of Acute Ischemic Stroke: Results from the SWIFT PRIME Randomized Controlled Trial” Goyal et al., Radiology, 2016.
“Regional Comparison of Multiphase Computed Tomographic Angiography and Computed Tomographic Perfusion for Prediction of Tissue Fate in Ischemic Stroke”, d'Esterre et al., Stroke 2017.

1. A method of imaging the brain within a patient diagnosed as potentially suffering a stroke, the method for deriving information about blood flow within the brain the method comprising the steps of:
a) obtaining a non-contrast rotational angiography scan of the patient's brain in the absence of contrast using a rotational angiography scanner, wherein the non-contrast rotational angiography scan is the first imaging scan of the patient's brain after a potential stroke, and wherein the rotational angiography scanner is configured to enable subsequent imaging of endovascular procedures;
b) injecting a bolus of contrast agent into the patient;
c) obtaining contrast rotational angiography scan of the patient's brain using the rotational angiography scanner at a time period after the bolus has been injected.

2. The method of claim 1, wherein the method comprises, at regular intervals, obtaining n contrast rotational angiography scans of the patient's brain, wherein n is between 2 and 6.

3. The method of claim 1, wherein the method comprises displaying the obtained sets of rotational angiography images as a time-sequenced series of images.

4. The method of claim 1, wherein the time intervals between successive sets of rotational angiography images is selected based on the anticipated flow rate of contrast agent through the patient.

5. The method of claim 1, wherein the brain is automatically divided up into a number of zones of interest by identifying pre-determined anatomical zones.

6. The method of claim 1, wherein the method further comprises the step of:
identifying collateral vessels facilitating collateral blood flow within the contrast rotational angiography scan based on: opacity of the vessels;
position of the vessels; and
the time delay between the bolus injection and the contrast rotational angiography scan.

7. The method of claim 1, wherein the method further comprises the step of superimposing data from the multiple rotational angiography scans and allocating colour based on delay of opacification.

8. The method of claim 1, wherein the method further comprises of evaluating the change in density at the level of each voxel of brain tissue and using the time-contrast curve to create maps which shows the tissue at risk due to the intracranial occlusion.

9. The method of claim 1, wherein the different rotational angiography scans correspond to different radiation exposures, and the method comprises using fixed elements from the scan with the highest radiation dose to increase the signal to noise ratio of at least one scan with the lower radiation dose.

10. The method of claim 1, wherein the method comprises:
calculating the rate of collateral flow based on the opacity profile of one or more identified collateral vessels recorded over the time period of the contrast rotational angiography scan.

11. The method of claim 10, wherein the method comprises:
identifying collateral blood vessels by identifying adjacent voxels in three-dimensional space which have a x-ray opacity exceeding a predetermined threshold.

12. The method of claim 10, wherein the method comprises:
defining an axial axis of each identified collateral vessel;
calculating the contrast profile based on aggregating x-ray opacity values of voxels at different axial positions along the axial axis of each identified collateral vessel.

13. The method of claim 10, wherein the method comprises:
calculating the x-ray opacity profile within an identified vessel at a point in time.

14. The method of claim 11, wherein the rate of opacification of the collateral vessel as the contrast comes into the collateral vessel is used to calculate flow rate within the collateral vessel.

15. The method of claim 11, wherein the clearing of contrast from the collateral vessel is used to calculate the flow rate within the collateral vessel.

16. The method of claim 1, wherein the change of density of each voxel of brain tissue is calculated and the rise and fall of the density is used to create maps which demonstrate the ischemic tissue.

17. The method of claim 11, wherein the combination of calculated flow rate within the collaterals and the voxel based map of the ischemic tissue is used to calculate and differentiate between tissue at risk and tissue that is already dead.

18. The method of claim 1, further comprising:
using a non-transitory computer readable medium encoded with instructions to analyse the contrast rotational angiography images according to the following:
map a plurality of zones of interest;
analyze each zone of interest to assign a collateral value to each zone of interest where an assigned collateral value represents relative viability of collaterals within that zone and where the assigned collateral value represents the total collateral blood flow into the zone;
calculating a secondary score based on a cumulative total of values from the assigned collateral values.

19. The method of claim 18, wherein the zones of interest and the secondary score correspond to the ASPECTS protocol.

20. The method of claim 18, wherein the zones of interest and the secondary zones correspond to a modified ASPECTS protocol focusing on the cortical regions.

21. The method of claim 1, wherein the method comprises:
identifying multiple fixed points associated with the patient's head during each rotational angiography scan;
identifying and correcting for movement of these fixed points during the scan.

22. The method of claim 21, wherein the multiple fixed points comprise inherent features of the patients head.

23. The method of claim 21, wherein the multiple fixed points comprise external markers affixed to the patients head.

24. The method of claim 1, wherein the method comprises:
obtaining one of the rotational angiography scans at a higher x-ray intensity than at least one other rotational angiography scan; and
using data from the higher x-ray intensity scan to constrain post-processing of the at least one other rotational angiography scan.

25. A system for imaging the brain within a patient diagnosed as potentially suffering a stroke, the system comprising:
a rotational angiography scanner, wherein the scanner is configured to
obtain a non-contrast rotational angiography scan of the patient's brain in the absence of contrast using a rotational angiography scanner, wherein the non-contrast rotational angiography scan is the first imaging scan of the patient's brain after a potential stroke, and wherein the rotational angiography scanner is configured to enable subsequent imaging of endovascular procedures; and
obtain contrast rotational angiography scan of the patient's brain using the rotational angiography scanner at a time period after the bolus has been injected.

26. A computer program for imaging the brain within a patient diagnosed as potentially suffering a stroke, the computer program being configured, when run on a computer configured to control a rotational angiography scanner, to:
obtain a non-contrast rotational angiography scan of the patient's brain in the absence of contrast using a rotational angiography scanner, wherein the non-contrast rotational angiography scan is the first imaging scan of the patient's brain after a potential stroke, and wherein the rotational angiography scanner is configured to enable subsequent imaging of endovascular procedures;
obtain contrast rotational angiography scan of the patient's brain using the rotational angiography scanner at a time period after a bolus has been injected.
